(19) United States
(12) Patent Application Publication
(10) Pub. No.: US 2021/0215635 A1
(43) Pub. Date: Jul. 15, 2021

(54) HEXAGONAL FLOW CELL WITH INTEGRATED CMOS SENSOR ARRAY

(71) Applicant: Roswell Biotechnologies, Inc., San Diego, CA (US)

(72) Inventors: Calvin James Gardner, San Diego, CA (US); Anders Lassen, San Diego, CA (US); Eskild Hansen, San Diego, CA (US); Barry Merriman, San Diego, CA (US); Paul Mola, San Diego, CA (US)

(73) Assignee: Roswell Biotechnologies, Inc., San Diego, CA (US)

(21) Appl. No.: 16/741,278

(22) Filed: Jan. 13, 2020

(51) Int. Cl. G01N 27/414; C12Q 1/6869

(52) U.S. Cl. CPC G01N 27/4148; C12Q 1/6869

(57) ABSTRACT

A hexagonal flow cell is configured with a CMOS sensor array to be integrated therein. Via use of the flow cell, improved DNA sequencing results may be achieved.

### TECHNICAL FIELD

The present disclosure relates to flow cells, and in particular to flow cells having sensor arrays integrated therein.

### BACKGROUND

Molecular analysis has received an increasing amount of attention in various fields such as precision medicine or nanotechnology. One example includes the analysis of molecules for sequencing genomes. The seminal work of Avery in 1946 demonstrated that DNA was the material that determined traits of an organism. The molecular structure of DNA was then first described by Watson and Crick in 1953, for which they received the 1962 Nobel Prize in Medicine. This work made it clear that the sequence of chemical letters (bases) of the DNA molecules encode the fundamental biological information. Since this discovery, there has been a concerted effort to develop means to actually experimentally measure this sequence. The first method for systematically sequencing DNA was introduced by Sanger in 1978, for which he received the 1980 Nobel Prize in Chemistry.

A basic method for sequencing a genome was automated in a commercial instrument platform in the late 1980's, which ultimately enabled the sequencing of the first human genome in 2001. This was the result of a massive public and private effort taking over a decade, at a cost of billions of dollars, and relying on the output of thousands of dedicated DNA sequencing instruments. The success of this effort motivated the development of a number of “massively parallel” sequencing platforms with the goal of dramatically reducing the cost and time required to sequence a human genome. Such massively parallel sequencing platforms generally rely on processing millions to billions of sequencing reactions at the same time in highly miniaturized microfluidic formats. The first of these was invented and commercialized by Rothberg in 2005 as the 454 platform, which achieved thousand fold reductions in cost and instrument time. However, the 454 platform still required approximately a million dollars and took over a month to sequence a genome.

The '454 platform was followed by a variety of other related techniques and commercial platforms. This progress lead to the realization of the long-sought “$1,000 genome” in 2014, in which the cost of sequencing a human genome at a service lab was reduced to approximately $1,000, and could be performed in several days. However, the highly sophisticated instrument for this sequencing cost nearly one million dollars, and the data was in the form of billions of short reads of approximately 100 bases in length. The billions of short reads often further contained errors so the data required interpretation relative to a standard reference genome with each base being sequenced multiple times to assess a new individual genome.

Thus, further improvements in quality and accuracy of sequencing, as well as reductions in cost and time are still needed. This is especially true to make genome sequencing practical for widespread use in precision medicine, where it is desirable to sequence the genomes of millions of individuals with a clinical grade of quality. Accordingly, improved sequencing techniques, components, systems, and methods remain desirable.

### BRIEF DESCRIPTION OF THE DRAWINGS

The features and advantages of the embodiments of the present disclosure will become more apparent from the detailed description set forth below when taken in conjunction with the drawings. The drawings and the associated descriptions are provided to illustrate embodiments of the disclosure and not to limit the scope of what is claimed.

FIGS. 1 through 15 illustrate components of an exemplary hexagonal flow cell in accordance with various exemplary embodiments.

FIGS. 16A through 16Y illustrate components of an exemplary hexagonal flow cell, and exemplary use thereof, in accordance with various exemplary embodiments.

FIGS. 17 through 20 illustrate components of an exemplary hexagonal flow cell in accordance with various exemplary embodiments.

FIGS. 21A through 21D illustrate various configurations for an exemplary hexagonal flow cell in accordance with various exemplary embodiments.

FIGS. 22 through 35 illustrate components of various exemplary hexagonal flow cells and associated systems in accordance with various exemplary embodiments.

FIGS. 36A through 36G illustrate views showing design elements of an exemplary hexagonal flow cell in accordance with various exemplary embodiments.

FIGS. 37A and 37B illustrate cutaway views of the hexagonal flow cell design of FIGS. 36A through 36G in accordance with various exemplary embodiments.

### DETAILED DESCRIPTION

In the following detailed description, numerous specific details are set forth to provide a full understanding of the present disclosure. It will be apparent, however, to one of ordinary skill in the art that the various embodiments disclosed may be practiced without some of these specific details. In other instances, well-known structures and techniques have not been shown in detail to avoid unnecessarily obscuring the various embodiments.

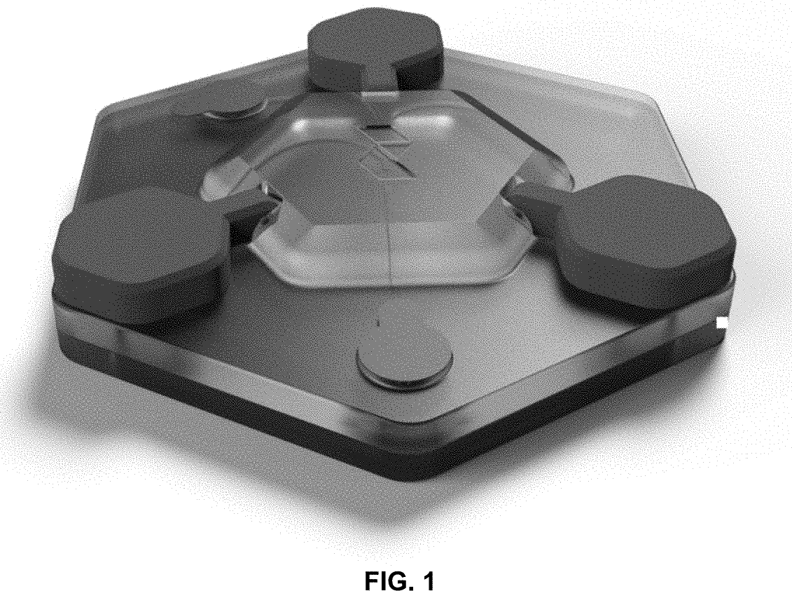

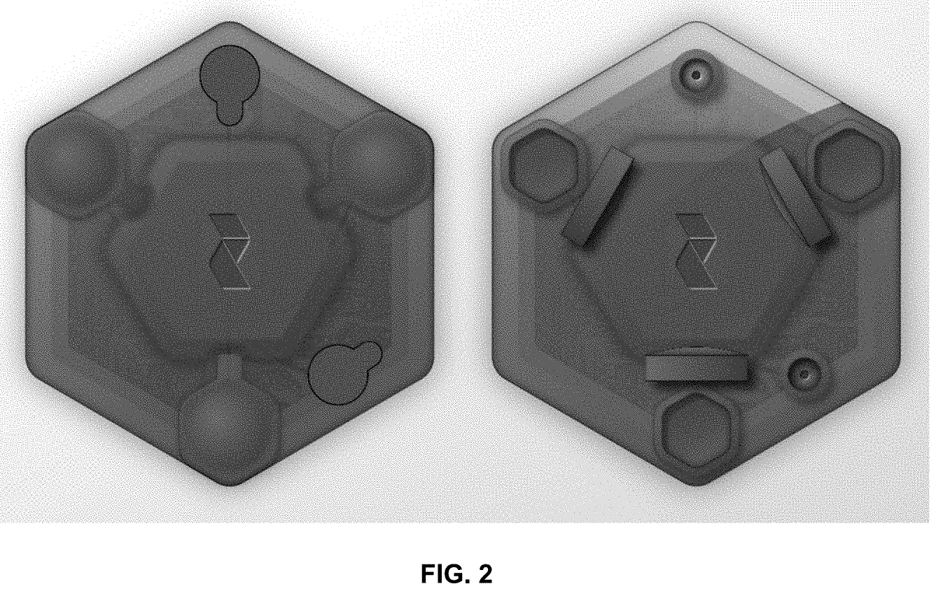

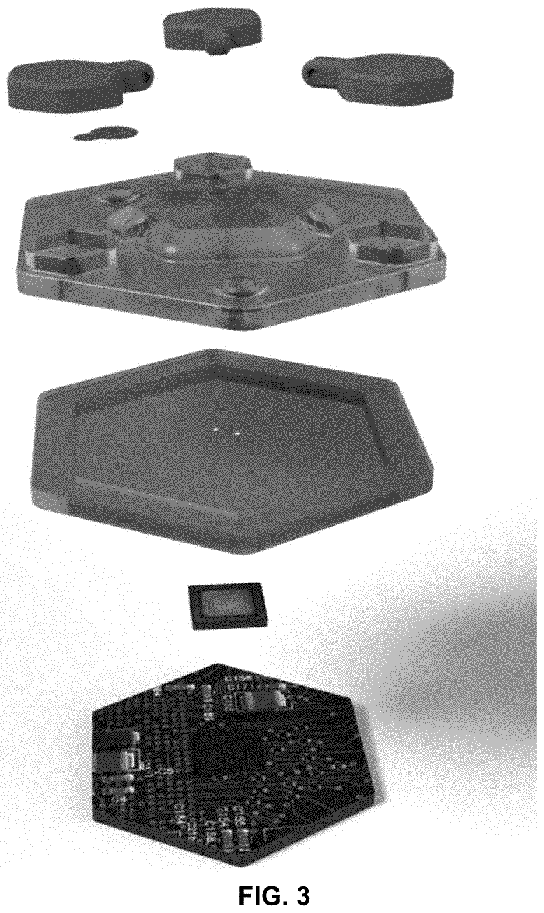

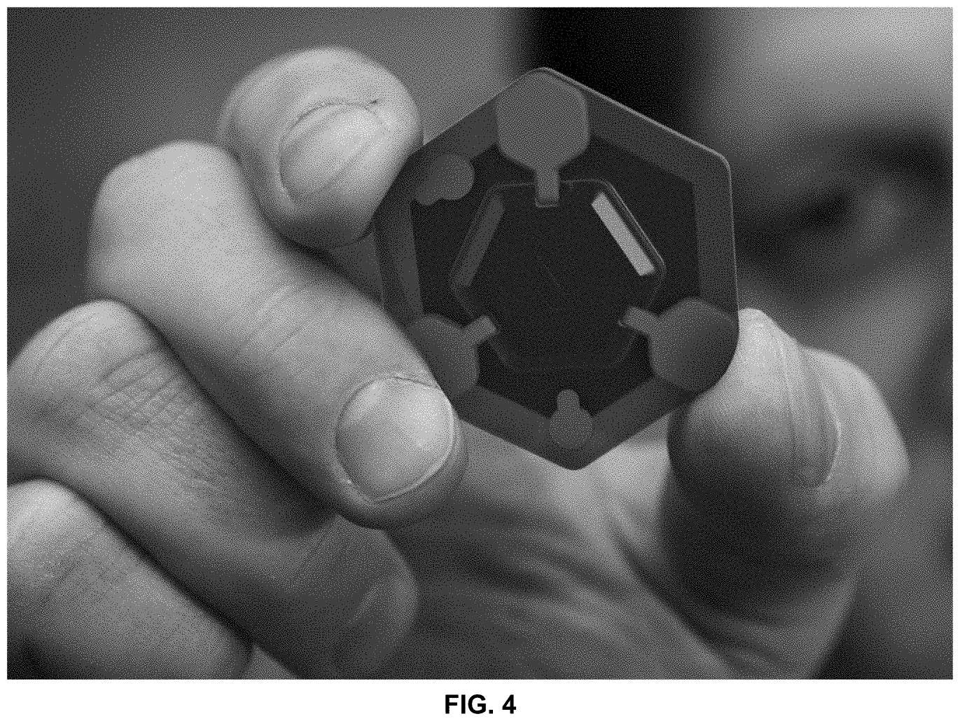

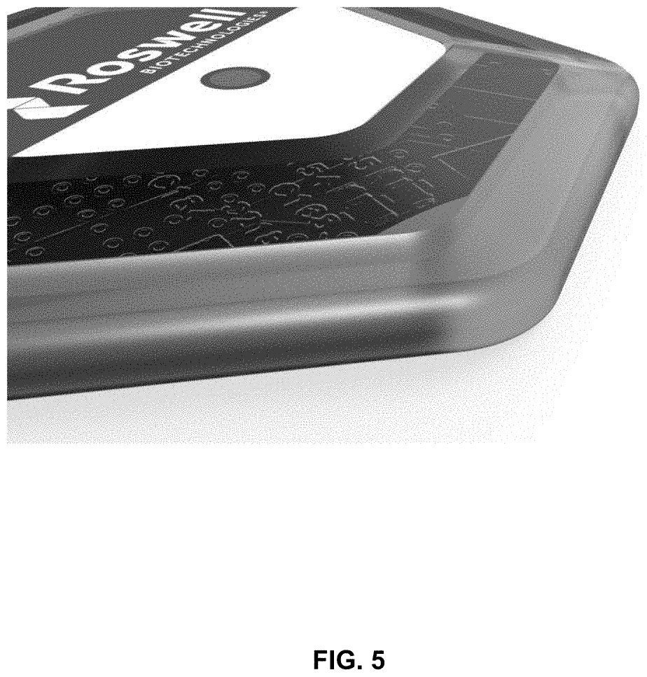

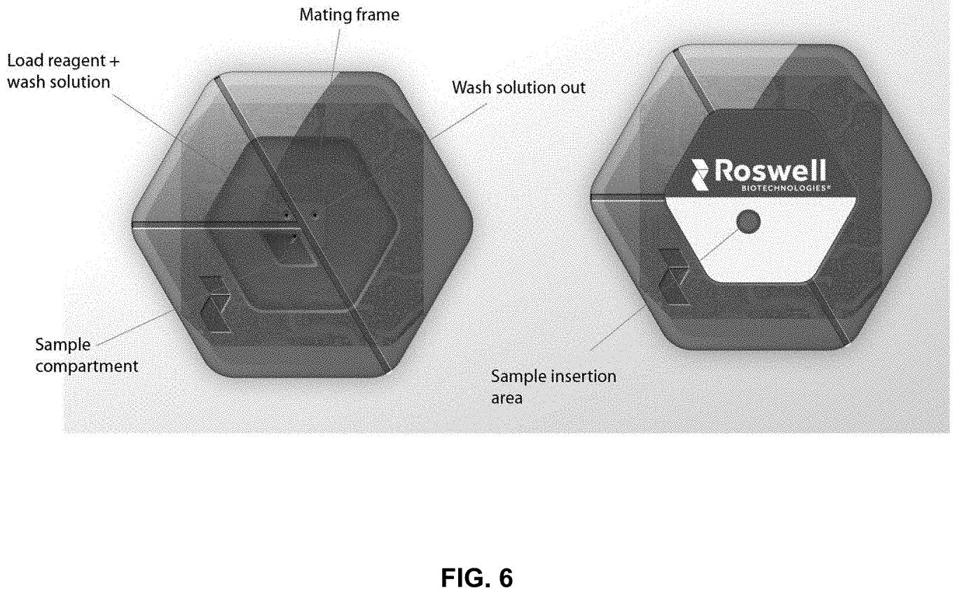

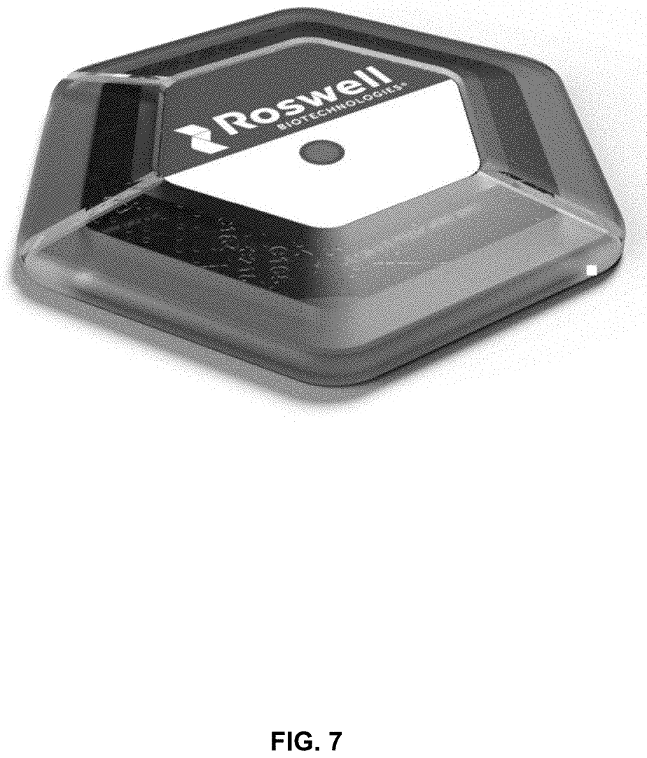

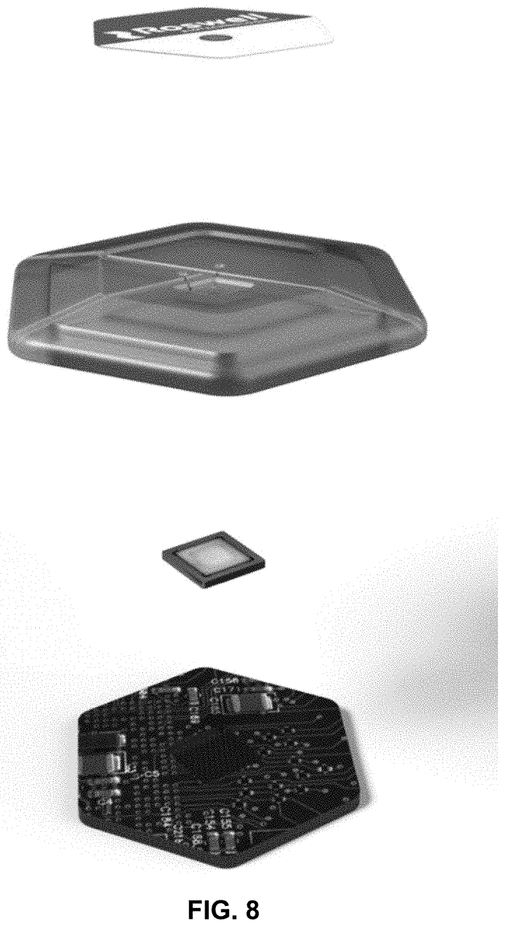

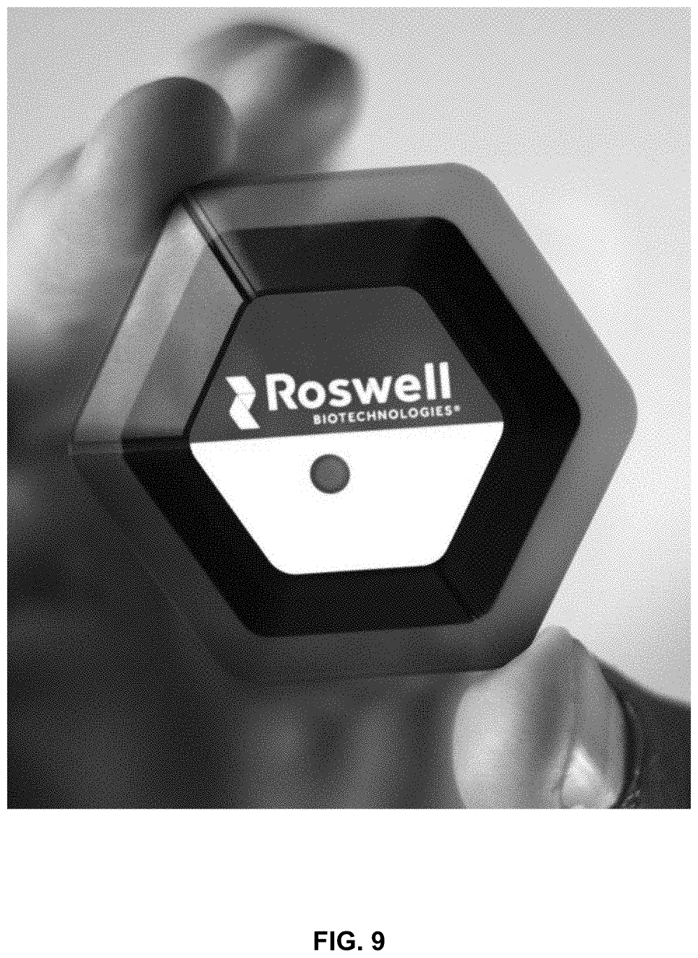

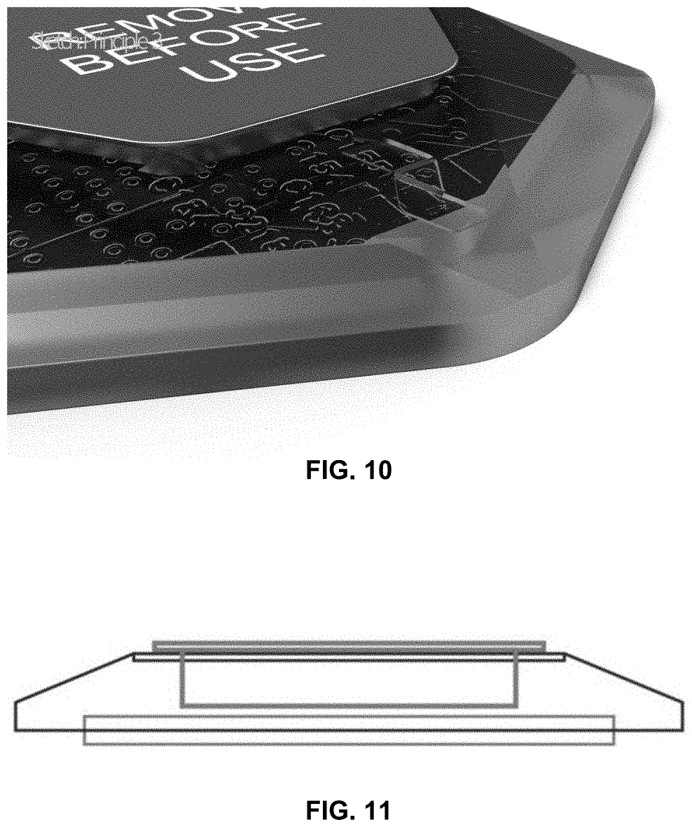

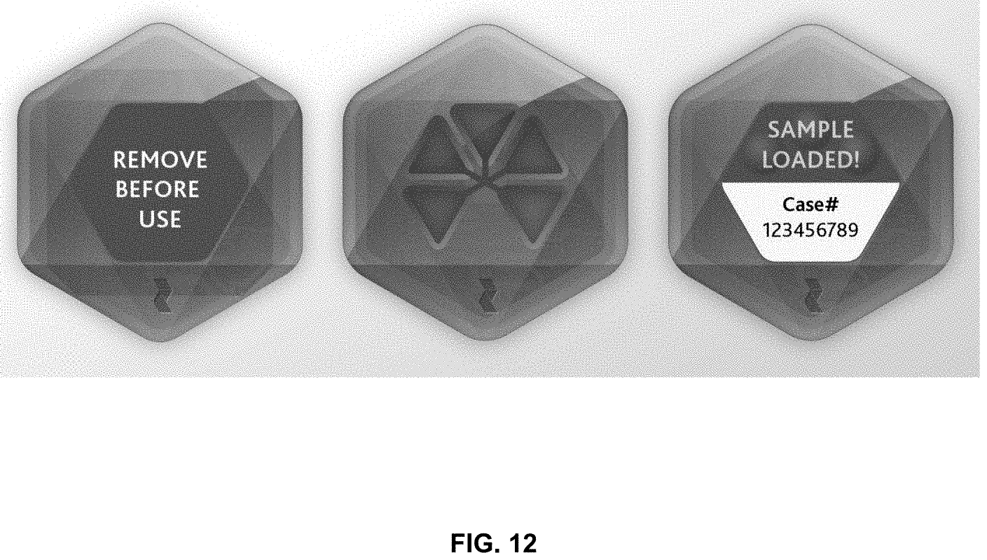

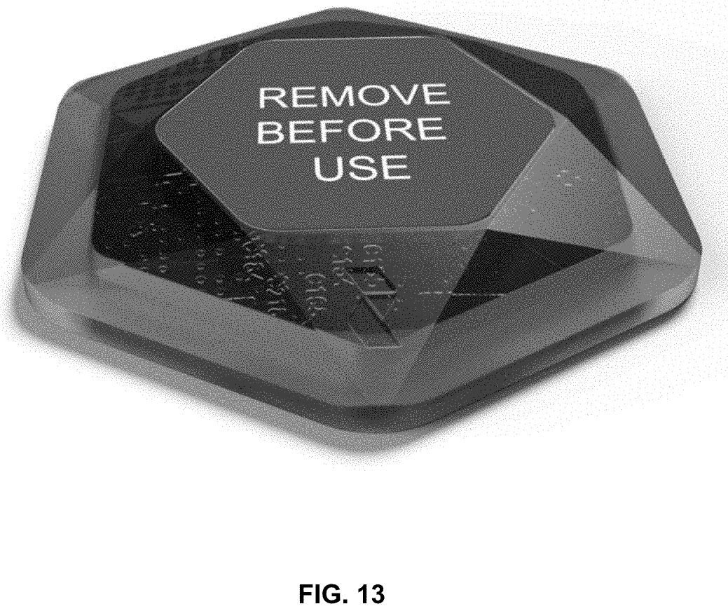

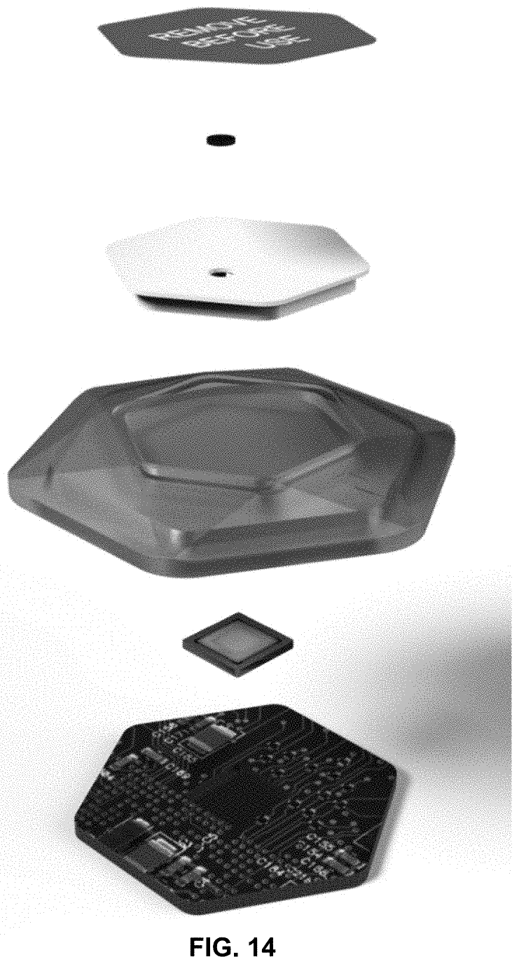

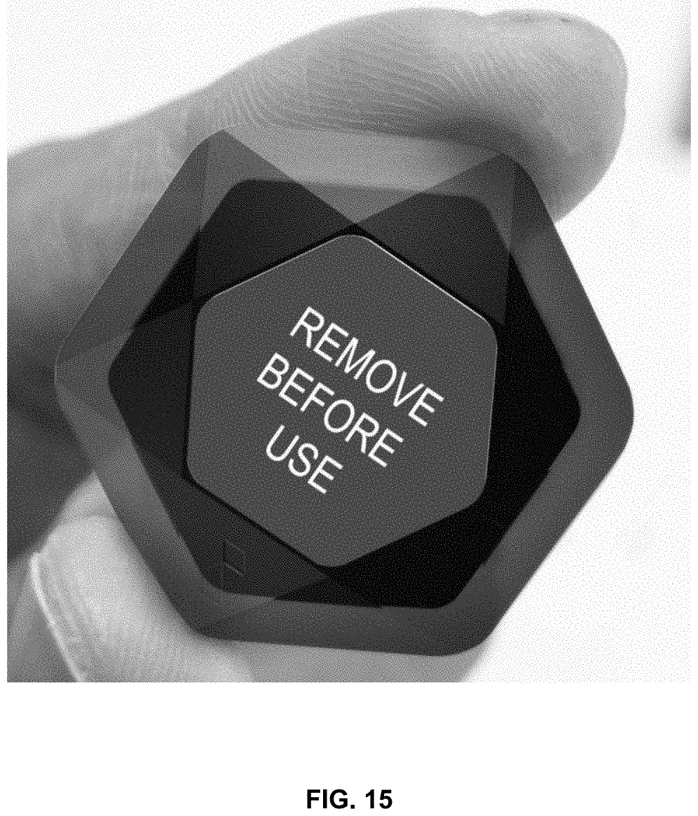

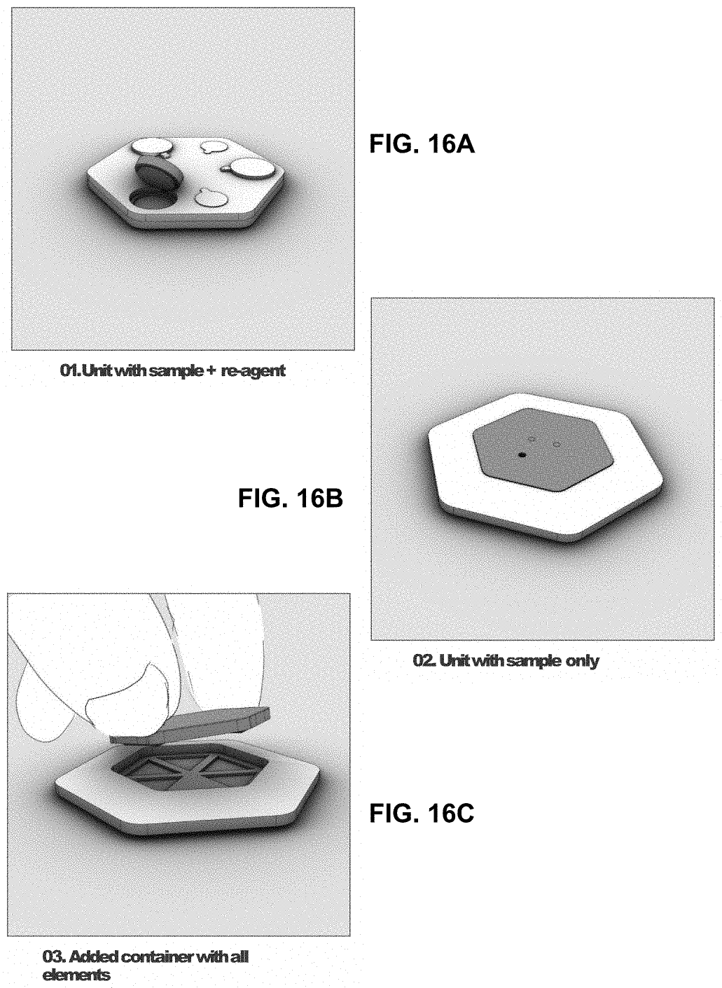

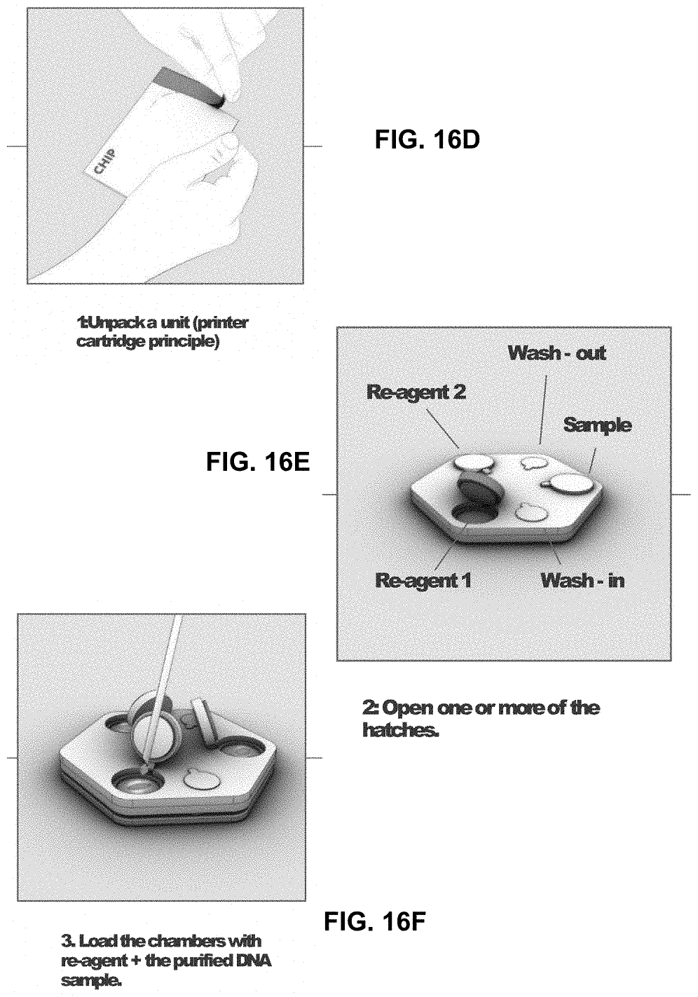

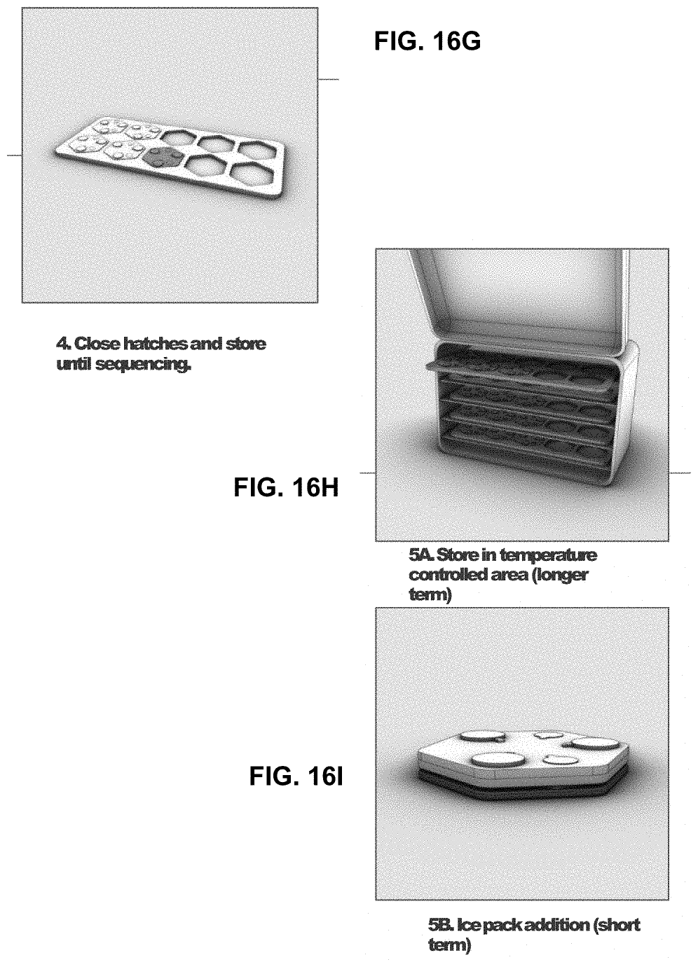

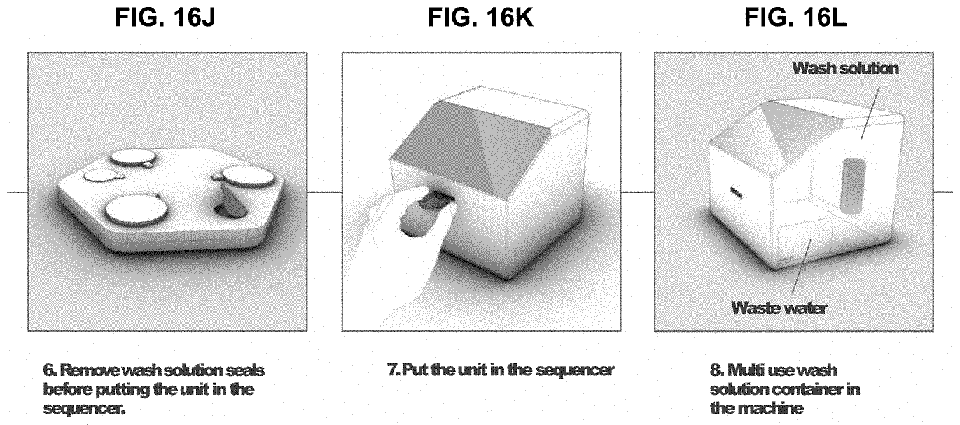

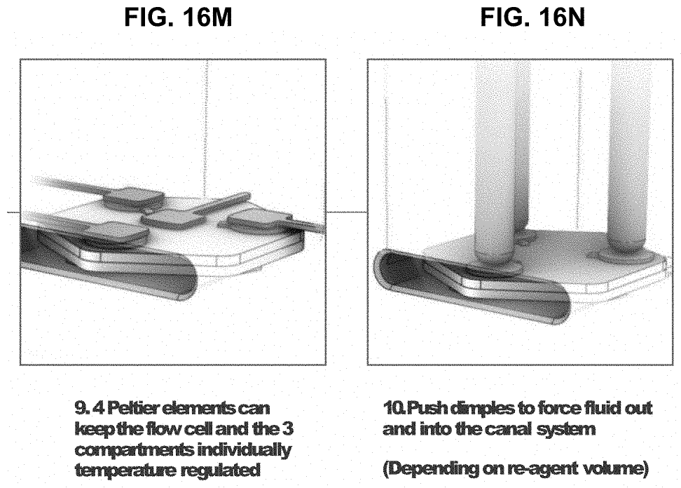

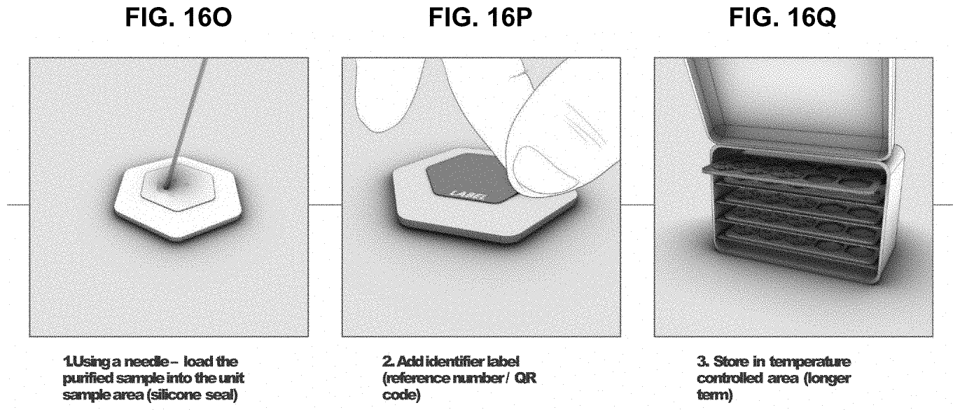

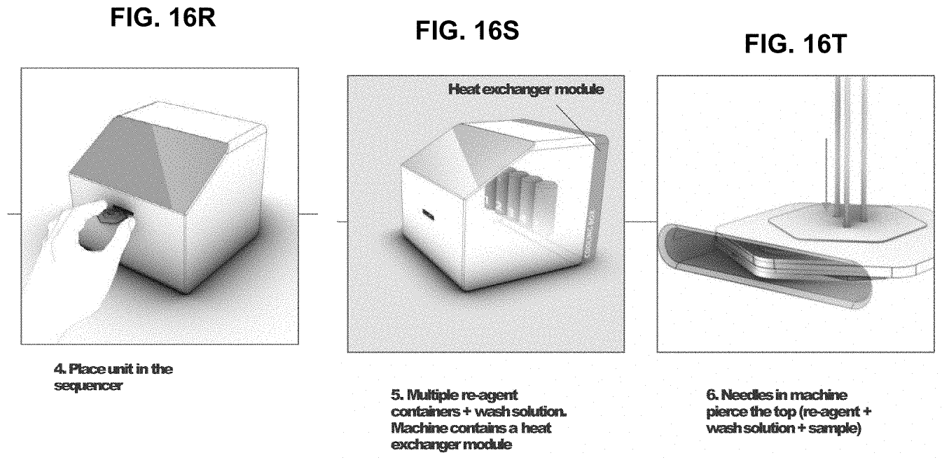

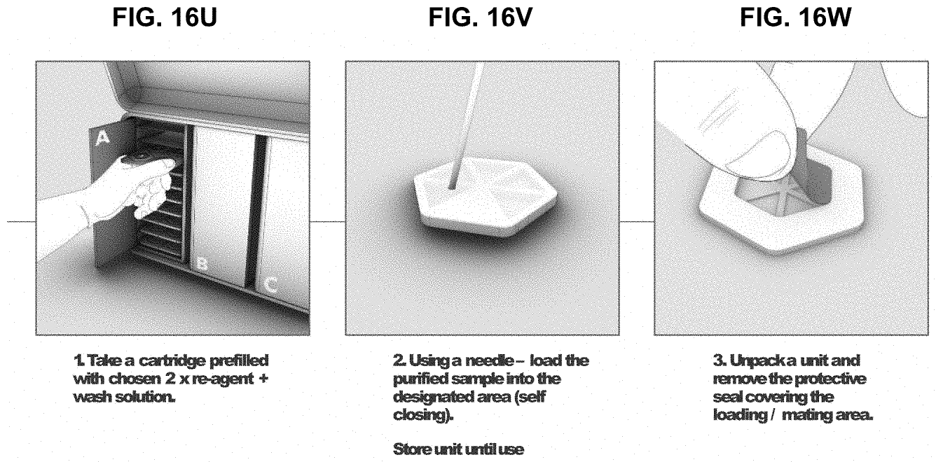

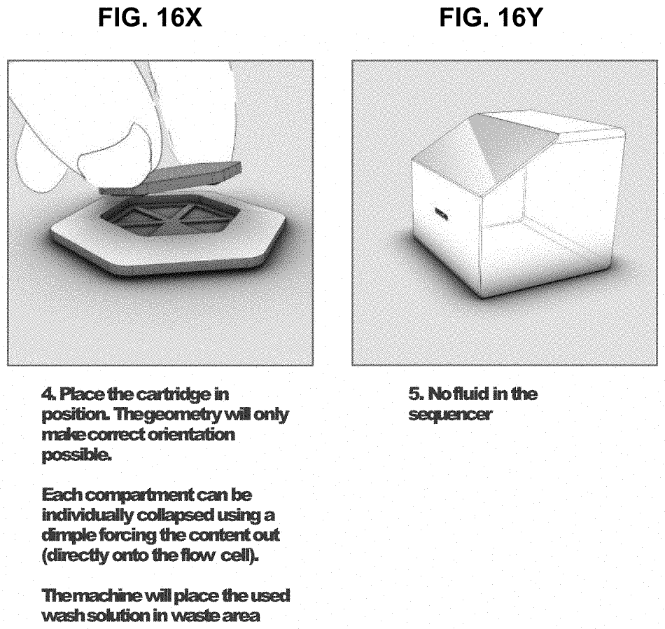

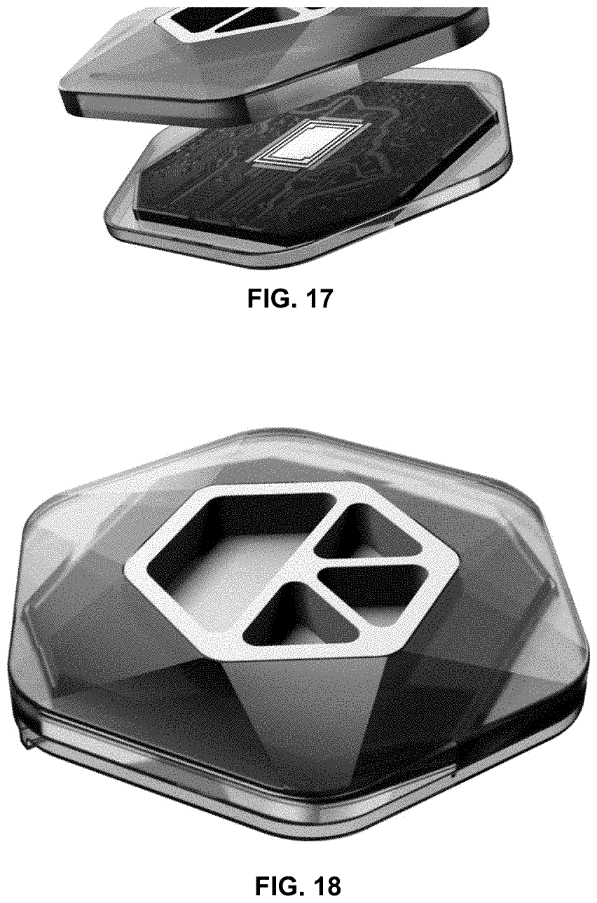

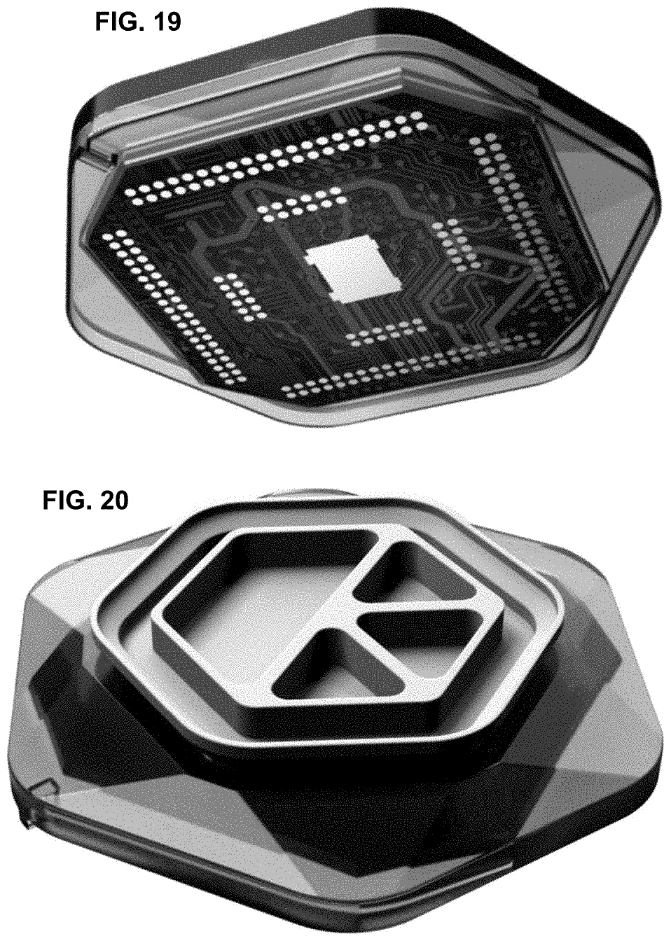

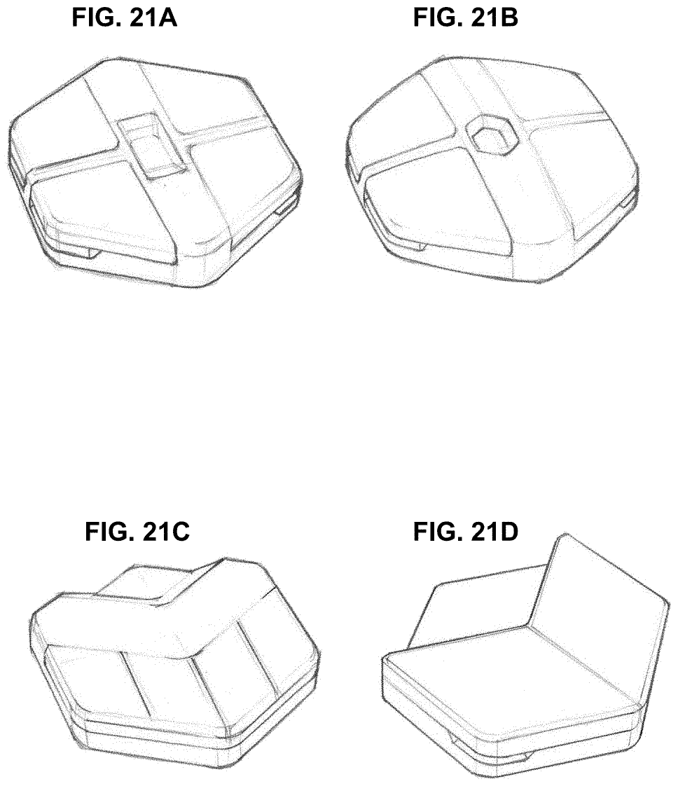

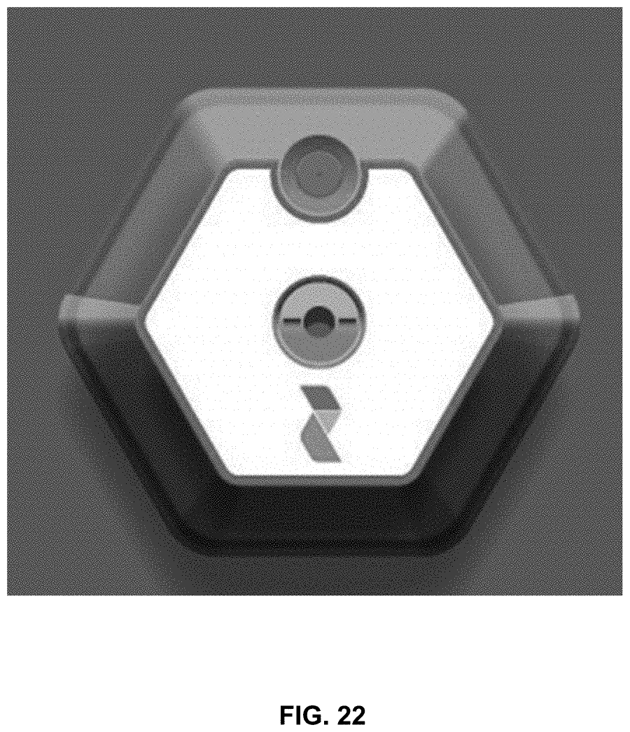

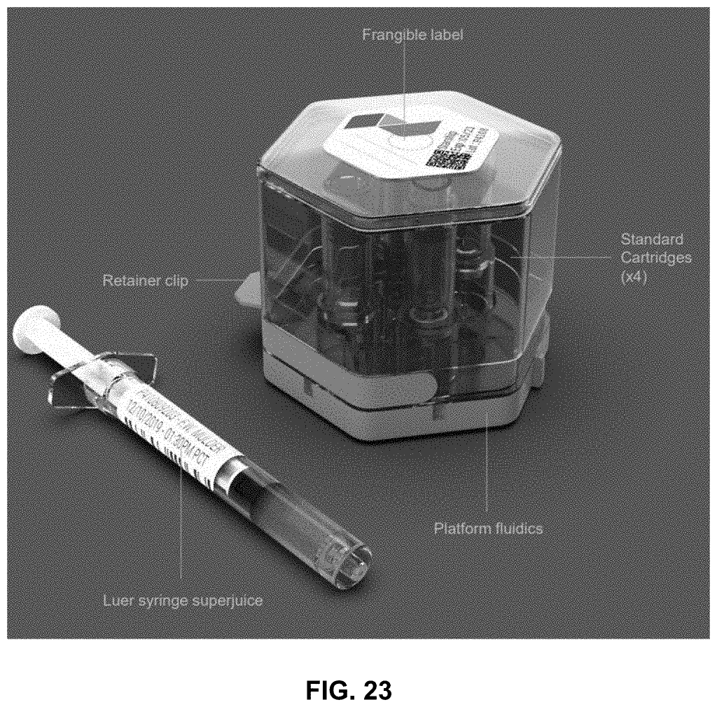

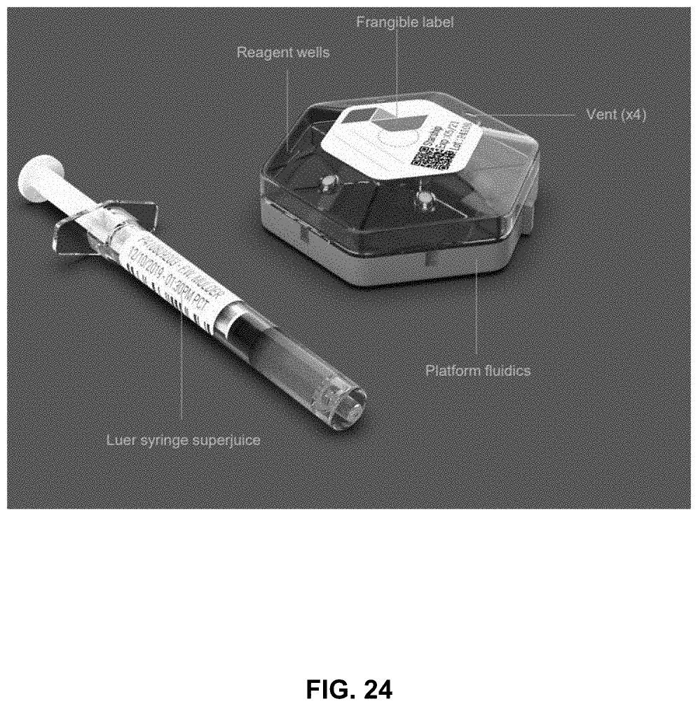

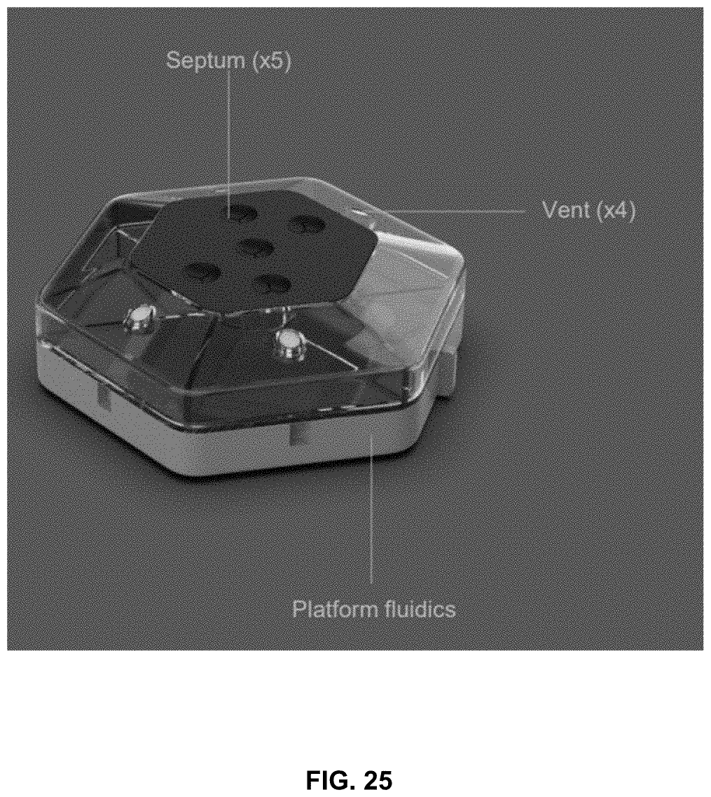

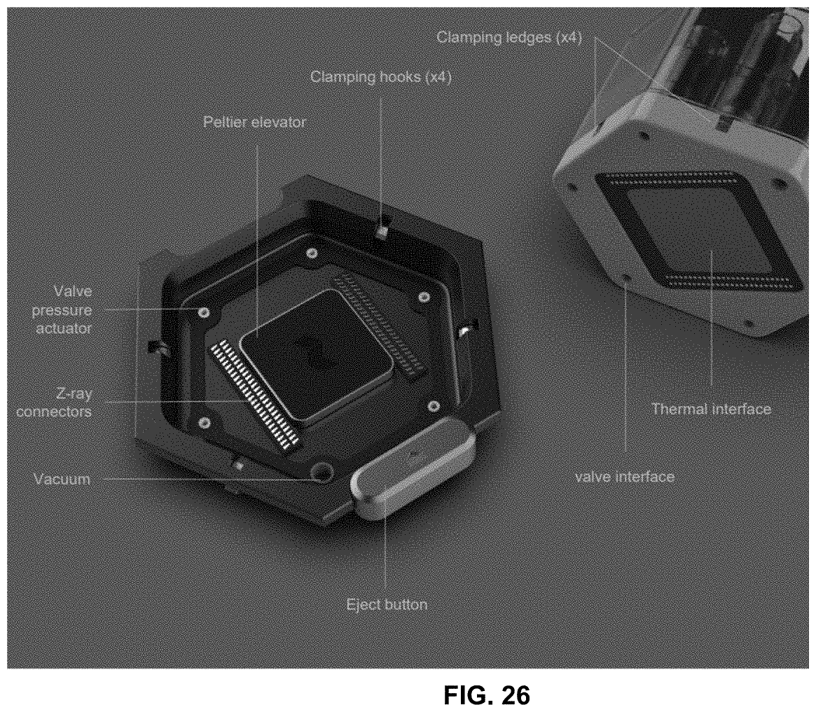

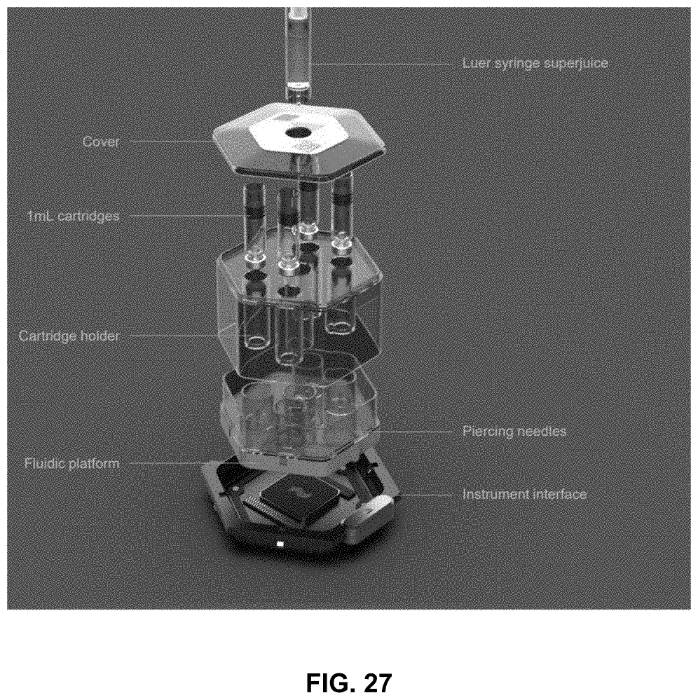

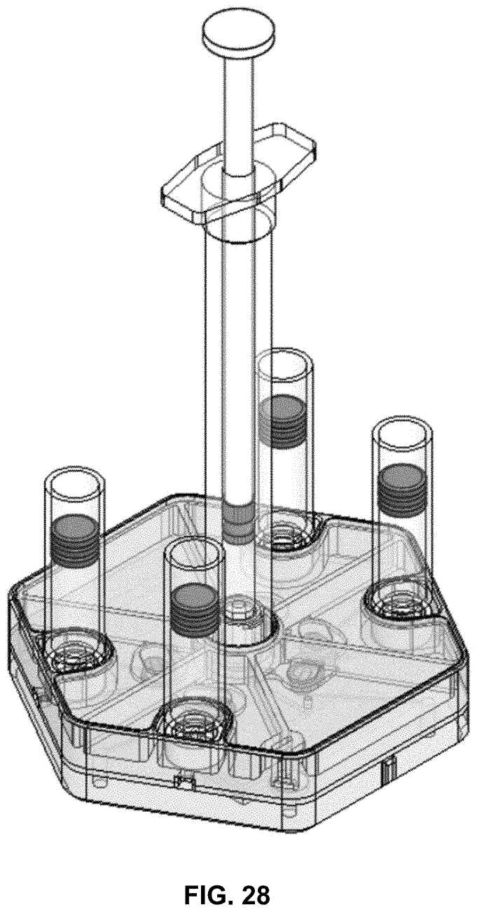

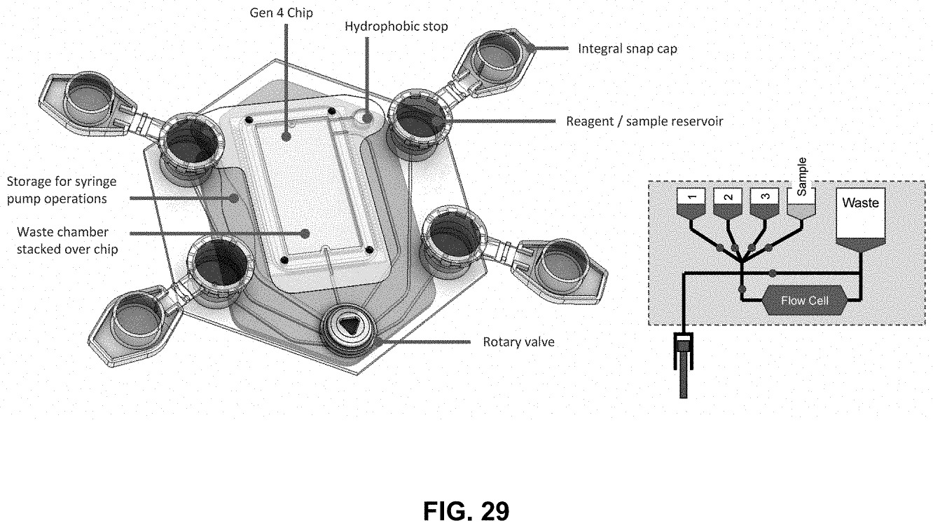

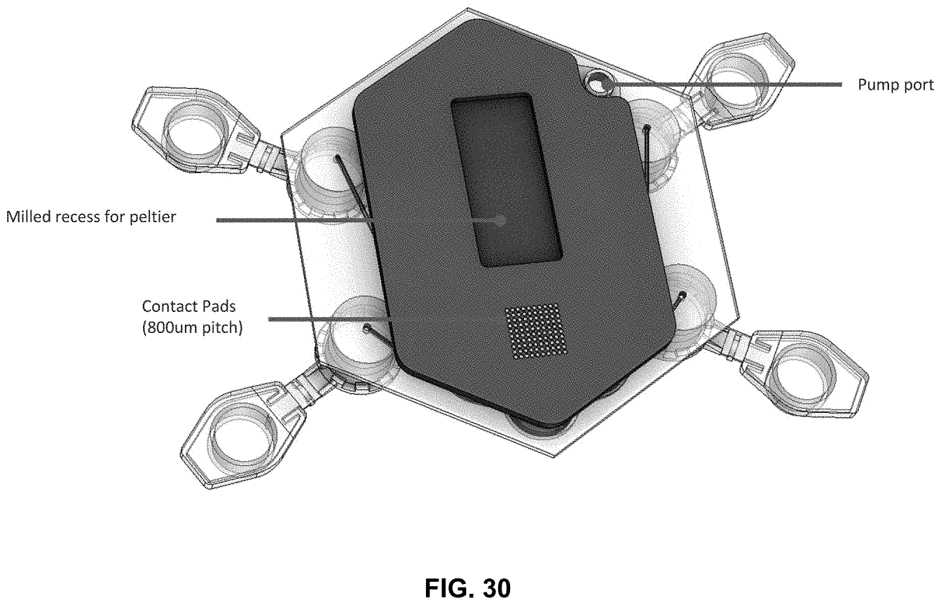

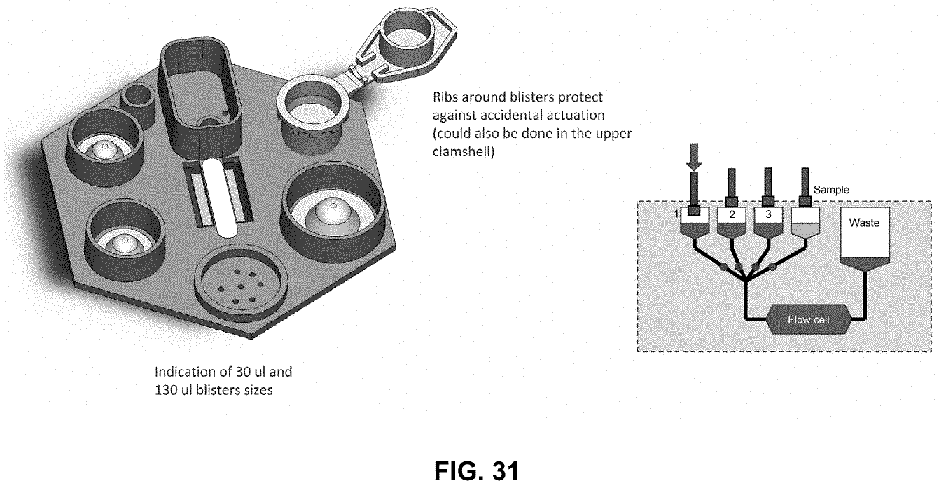

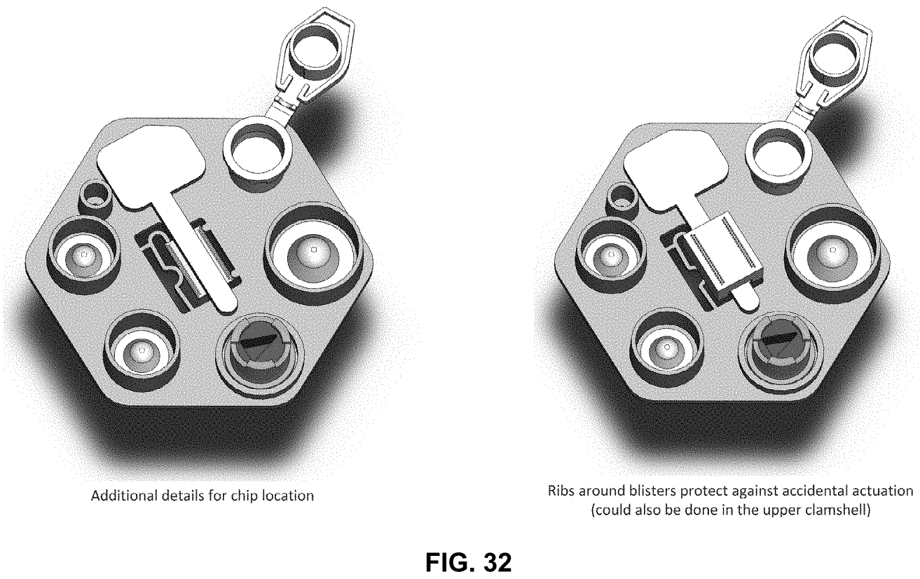

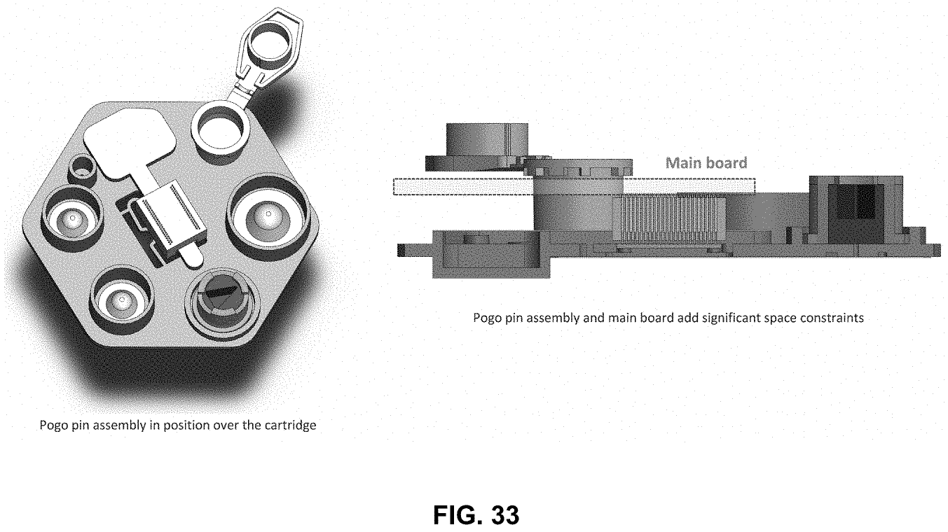

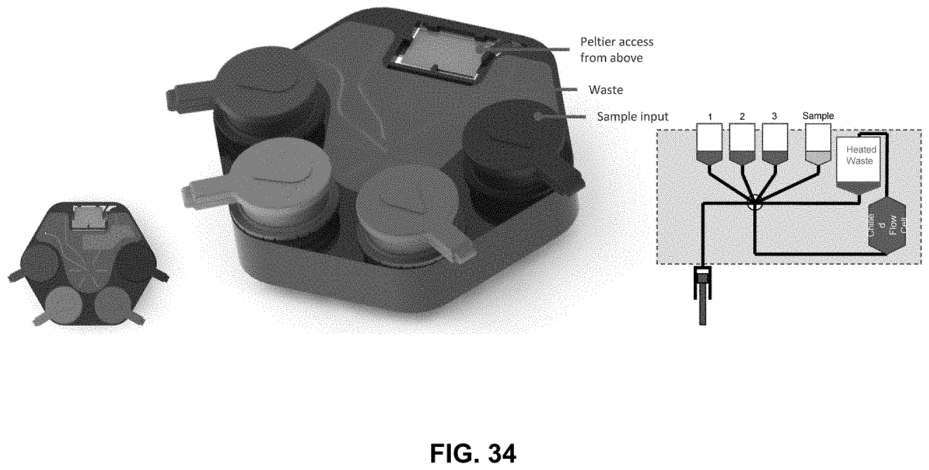

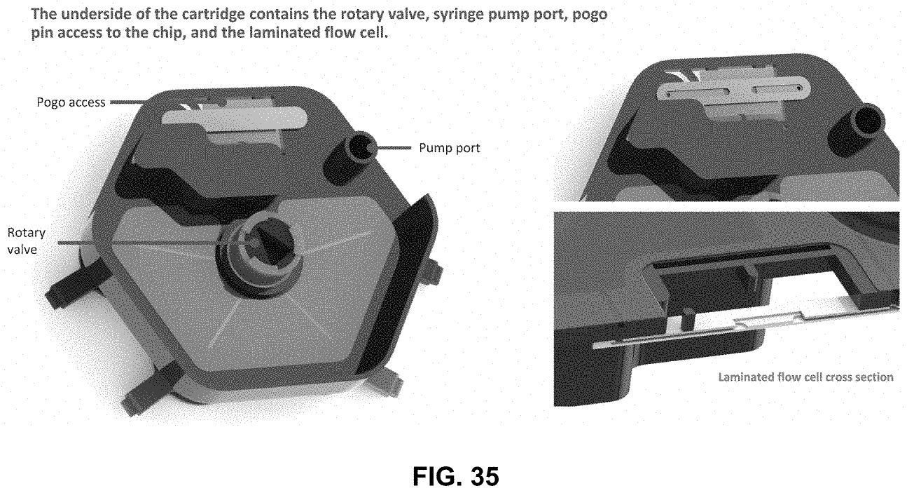

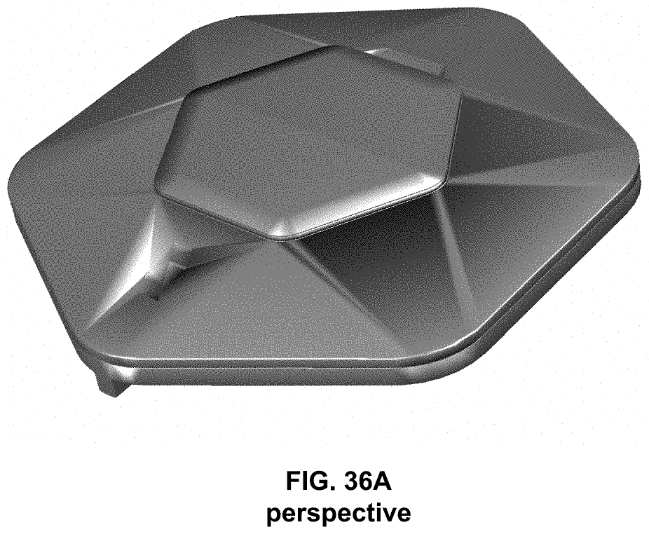

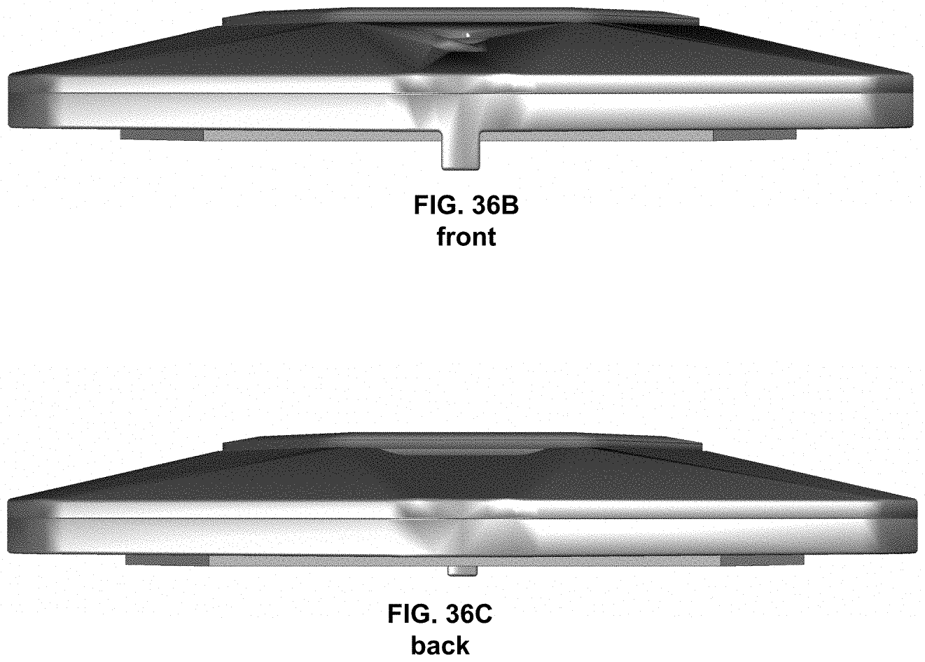

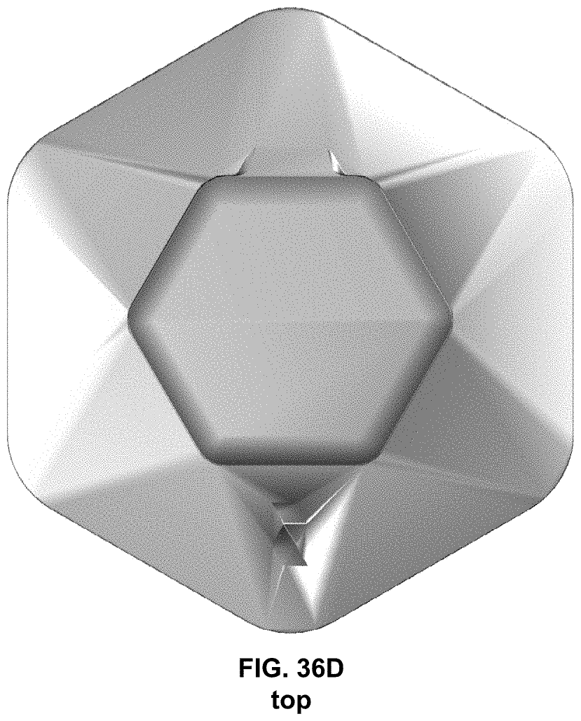

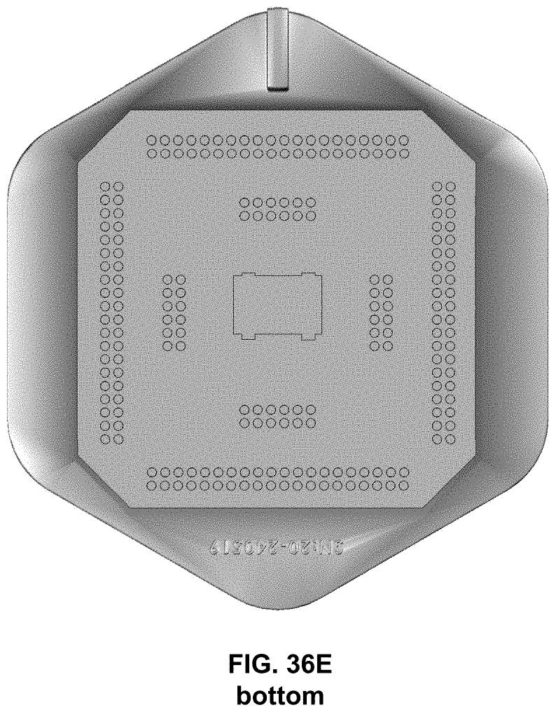

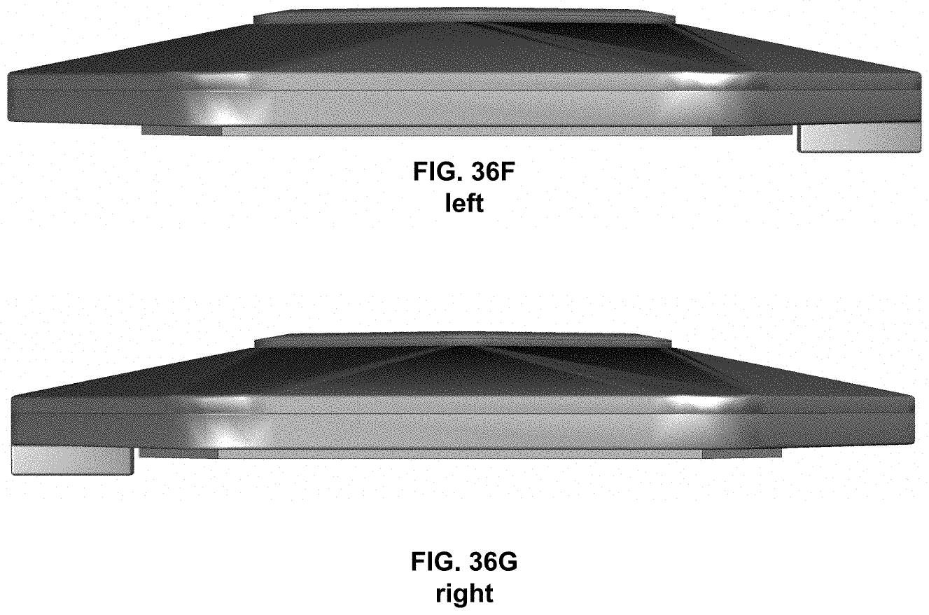

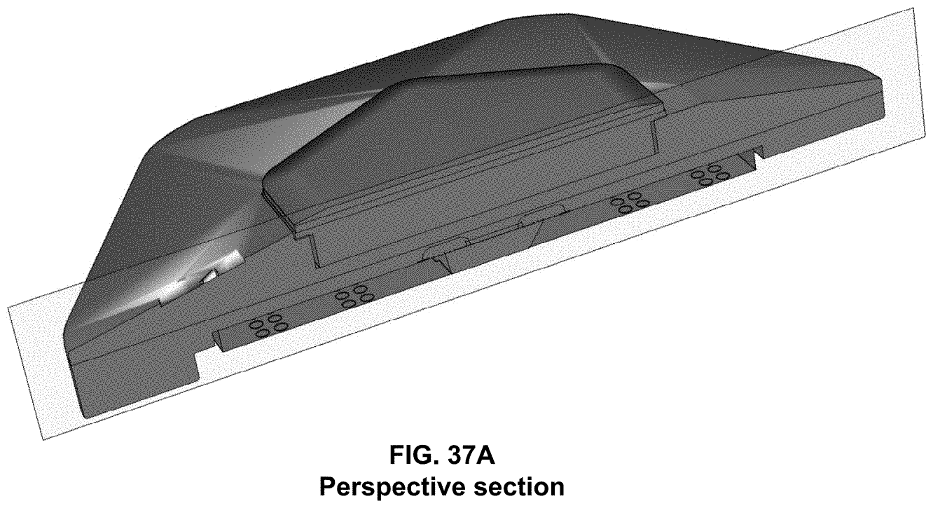

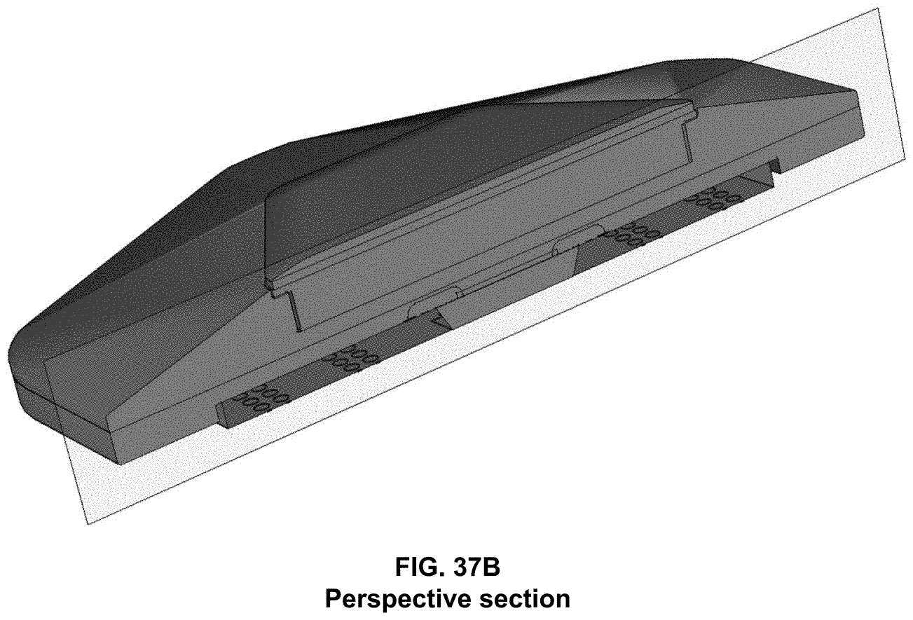

The views of FIGS. 1 through 37B illustrate various aspects of an exemplary hexagonal flow cell with an integrated CMOS sensor array in accordance with various exemplary embodiments. For example, various figures illustrate use of an exemplary hexagonal flow cell. Other figures illustrate components of an exemplary hexagonal flow cell. Still other figures illustrate ornamental design aspects of an exemplary hexagonal flow cell.

The foregoing description of the disclosed example embodiments is provided to enable any person of ordinary skill in the art to make or use the embodiments in the present disclosure. Various modifications to these examples will be readily apparent to those of ordinary skill in the art, and the principles disclosed herein may be applied to other examples without departing from the present disclosure. The described embodiments are to be considered in all respects only as illustrative and not restrictive, and the scope of the disclosure is therefore indicated by the following claims rather than by the foregoing description. All changes which come within the meaning and range of equivalency of the claims are to be embraced within their scope.

What is claimed is:

1. A hexagonal flow cell having an integrated CMOS sensor array, the hexagonal flow cell comprising:
a flow cell housing; and
a CMOS sensor array disposed within the flow cell housing.

2. The hexagonal flow cell of claim 1, wherein the flow cell housing is configured with a hexagonal shape.
